(19) United States
(12) Patent Application Publication
(10) Pub. No.: US 2008/0144178 A1
(43) Pub. Date: Jun. 19, 2008

(54) MICROSCOPE DRAPE LENS COVER SYSTEM AND ASSEMBLY METHOD

(75) Inventors: Mark S. Dillon, Columbus, MS (US); Ginger C. Pack-Walden, Columbus, MS (US); Tammy C. Adams, Columbus, MS (US); Youzhen Ding, Alpharetta, GA (US)

Correspondence Address: BAKER BOTTS L.L.P., 2001 ROSS AVENUE, SUITE 600, DALLAS, TX, 75201-2980, US

(73) Assignee: Microtek Medical, Inc., Columbus, MS (US)

(21) Appl. No.: 11/954,524

(22) Filed: Dec. 12, 2007

Related U.S. Application Data
(60) Provisional application No. 60/874,985, filed on Dec. 13, 2006.

(51) Int. Cl. A61B 19/08

(52) U.S. Cl. 359/510

(57) ABSTRACT

According to one embodiment of the invention, a microscope drape disposable lens cover system includes a coupling member configured to couple to an objective lens barrel of a microscope. A lens housing is coupled to the coupling member. The lens housing includes a transparent protective lens positioned such that a geometric normal of the transparent protective lens forms an angle with respect to an optical axis of an objective lens housed within the objective lens barrel when the housing is coupled to the objective lens barrel. At least a portion of a fitting is disposed between the coupling member and the lens housing. The fitting is frictionally retained adjacent the coupling member to prevent the rotation of the fitting relative to the coupling member but enables the rotation of lens housing with respect to the objective lens barrel coupling member.

### TECHNICAL FIELD OF THE INVENTION

The present invention relates generally to the field of medical drapes and, more particularly, to a microscope drape lens cover system and assembly method.

### BACKGROUND OF THE INVENTION

To minimize the risk of infection to surgical patients in an operating room or to protect medical equipment from a surgical field during surgery, drapes are often utilized. Drapes may be placed over a patient and/or medical equipment to form a sterile barrier, keeping any microorganisms and contaminants that may cause infections from migrating to and from exposed tissue and open wounds. For example, bodily fluids during surgery may settle on medical equipment, which then become contaminated and hazardous to those persons who must work with the equipment. Instead, the bodily fluids will ultimately settle on the drapes and not on the draped medical equipment.

The advancement of medical procedures has correspondingly created a demand for more advanced medical equipment. For example, the surgical microscope has become an integral part of an operating room. The surgical microscope can be ceiling mounted, wall mounted or mounted on a floor stand and typically may be raised or lowered and positioned over any part of a patient's body. The surgical microscope often has multiple eyepieces that permit the surgeon and others to simultaneously view the magnified area under the microscope's objective lens.

A microscope drape, used to create a sterile barrier, may be affixed to the microscope at the lens housing of the objective lens to orient the drape with respect to the remaining structure of the microscope. Other portions of the drape may be spread and positioned to cover the remainder of the microscope structure. In order to be able to protect the objective lens and still be able to see the surgical area, a transparent protective lens is usually associated with the device that couples the drape to the microscope. The transparent protective lens, depending on its positioning, may cause an undesirable glare to the user of the microscope, which may complicate the surgical procedure.

### SUMMARY OF THE INVENTION

According to one embodiment of the invention, a microscope drape disposable lens cover system includes a coupling member configured to couple to an objective lens barrel of a microscope. A lens housing is coupled to the coupling member. The lens housing includes a transparent protective lens positioned such that a geometric normal of the transparent protective lens forms an angle with respect to an optical axis of an objective lens housed within the objective lens barrel when the housing is coupled to the objective lens barrel. At least a portion of a fitting is disposed between the coupling member and the lens housing. The fitting is frictionally retained adjacent the coupling member to prevent the rotation of the fitting relative to the coupling member but enables the rotation of lens housing with respect to the objective lens barrel coupling member.

Embodiments of the invention provide a number of technical advantages. Embodiments of the invention may include all, some, or none of these advantages. A microscope drape lens cover, according to one embodiment, includes an angled transparent protective lens to substantially reduce or eliminate any glare that a user may encounter during surgery or other medical procedure. This angled transparent protective lens may be housed within a lens cover system that rotates, which further may enhance its glare reduction capabilities. Many known microscope designs have a wide variation in the placement of light sources relative to the microscope lens. Thus, certain embodiments may provide an advantage in that the rotatable transparent protective lens may be easily rotated to any angular position relative to the light source in order to alleviate glare.

Other technical advantages are readily apparent to one skilled in the art from the following figures, descriptions, and claims.

### BRIEF DESCRIPTION OF THE DRAWINGS

For a more complete understanding of the invention, and for further features and advantages, reference is now made to the following description, taken in conjunction with the accompanying drawings, in which:

FIG. 1 is a perspective view of a microscope drape coupled to a microscope using a drape lens cover system in accordance with one embodiment of the present invention;

FIG. 2 is an exploded, perspective view of a microscope drape lens cover system in accordance with one embodiment of the present invention;

FIG. 3 is a cross-sectional view illustrating the microscope drape lens cover system of FIG. 2 coupled to a microscope in accordance with one embodiment of the present invention; and

FIG. 4 is a cross-sectional view illustrating a microscope drape coupler in accordance with one embodiment of the present invention in accordance with another embodiment of the present invention.

### DETAILED DESCRIPTION OF EXAMPLE EMBODIMENTS OF THE INVENTION

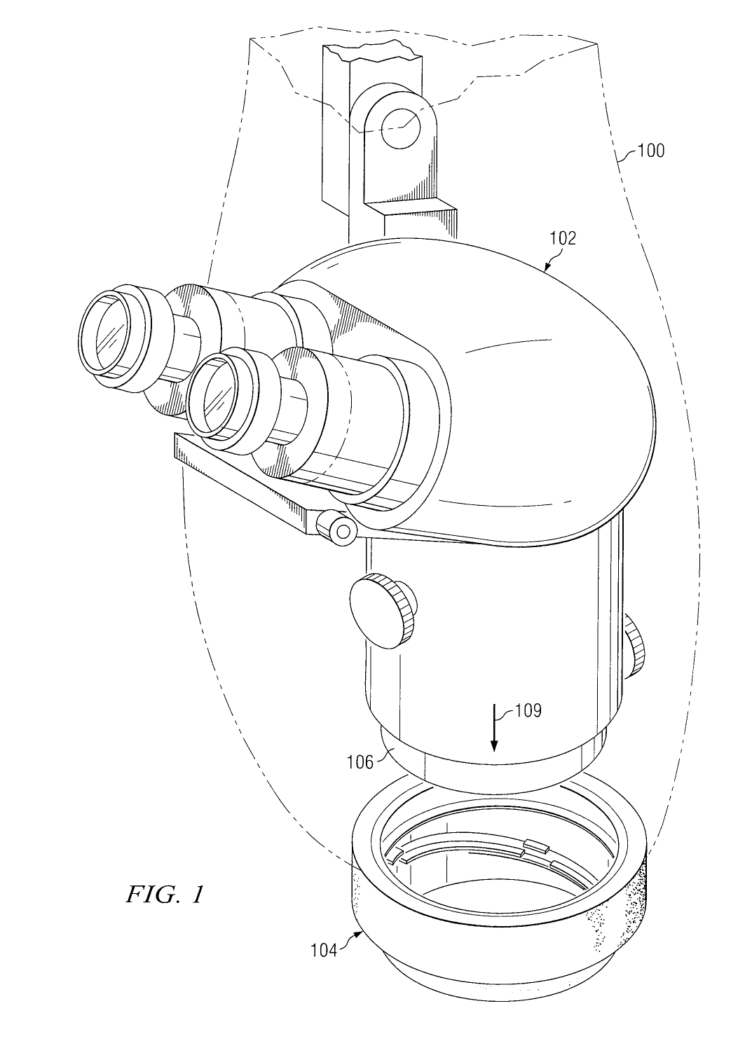

Example embodiments of the present invention and their advantages are best understood by referring now to FIGS. 1 through 4 of the drawings, in which like numerals refer to like parts.

FIG. 1 is a perspective view of a microscope drape 100 coupled to a microscope 102 using a drape lens cover system 104. Although the present invention contemplates any suitable medical instrument being protected by drape 100, the present invention is particularly suitable for surgical microscopes, such as microscope 102 illustrated in FIG. 1.

Drape 100 functions to generate a protective barrier between microscope 102 and its environment. For example, in an embodiment where microscope 102 is a surgical microscope, drape 100 protects microscope 102 from any bodily fluids, surgical fluids, and/or other materials during a surgical procedure from coming into contact with microscope 102. Conversely, any contaminants associated with microscope 102 are prevented from coming into contact with a patient during a surgical procedure. Any suitable drape 100 formed from any suitable material is contemplated by the present invention to cover microscope 102.

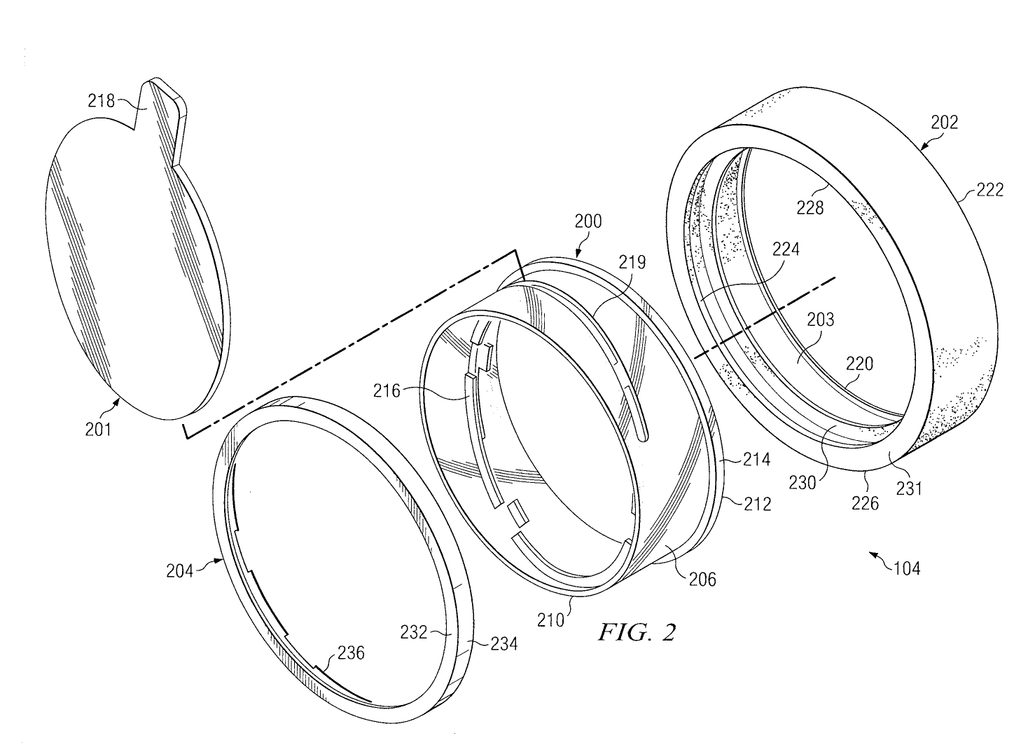

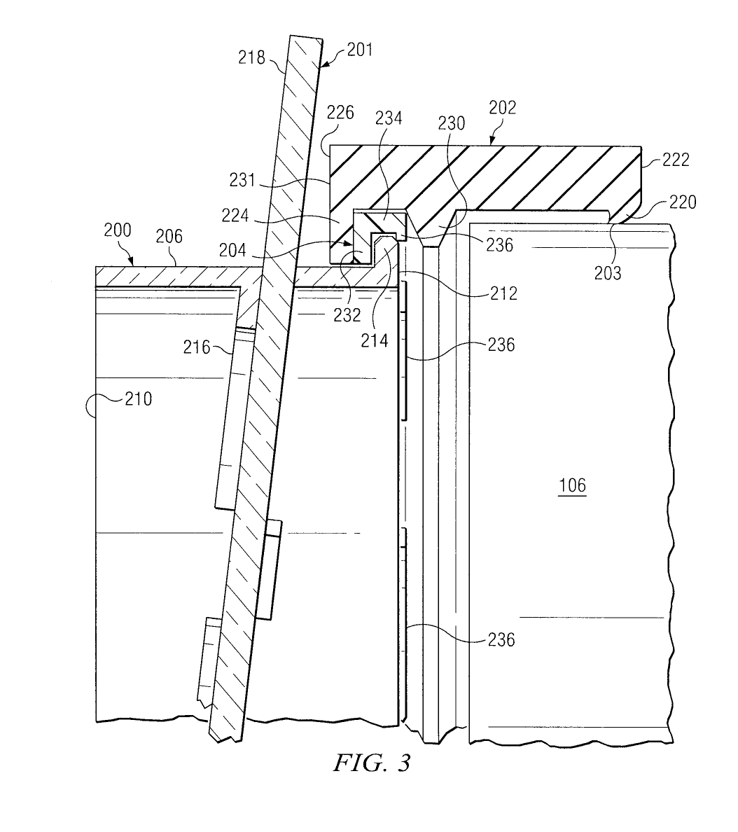

In the illustrated embodiment, drape lens cover system 104 couples to an objective lens barrel 106 of microscope 102; however, drape lens cover system 104 may couple to other portions of microscope 102 within the teachings of the present invention. Because there are many different types of microscopes available in the medical industry, many different sizes of objective lens barrels are encountered. Thus, a microscope drape lens cover system that fits the objective lens barrel of one microscope may not fit the objective lens barrel of another microscope. In addition, in order to be able to protect the objective lens of a microscope and still be able to see a surgical area, a transparent protective lens is typically associated with the drape lens cover system. The transparent protective lens, depending on its positioning, may cause an undesirable glare to the user of the microscope, which may complicate the surgical procedure. This transparent protective lens may also encounter bodily and/or surgical fluids during a surgical procedure, which means that the lens may need to be replaced during the surgical procedure. The present invention addresses these problems, and others, by providing drape lens cover system 104, as described in more detail below in conjunction with FIGS. 2 and 3.

FIG. 2 is an exploded, perspective view of microscope drape lens cover system 104 according to one embodiment of the present invention. The components are disassembled in order to reveal other components and features that are not visible when the drape lens cover system 104 is assembled. In the illustrated embodiment, drape lens cover system 104 includes a housing 200 having a removable transparent protective lens 201, an objective lens barrel coupling member 202 having an objective lens barrel aperture 203, and a fitting or track system 204 that is positioned between the housing 200 and the objective lens barrel coupling member 202. An advantage of the illustrated embodiment is that housing 200 is rotatably secured in objective lens barrel coupling member 202 by an outwardly extending flange 214 of housing 200 that lies adjacent to an inwardly extending retainer flange 224 formed on the objective lens barrel coupling member 202. When assembled, inwardly extending retainer flange 224 is configured to maintain outwardly extending flange 214 within the objective lens barrel coupling member 202. Fitting 204 secures lens housing 200 in objective lens barrel coupling member 202 and allows housing 200 to rotate with respect to objective lens barrel coupling member 202 and objective lens barrel 106.

As described in more detail below in conjunction with FIG. 3, housing 200 is rotatably coupled to objective lens barrel coupling member 202 in order to facilitate the rotation of transparent protective lens 201 to substantially reduce or eliminate any glare during a surgical procedure. Housing 200 may be any suitable size and shape and may be formed from any suitable material. In a particular embodiment, housing 200 may be made of plastic. Specific materials that may be used to form housing 200 include polyethylene, acrylonitrile butadiene styrene (ABS), nylon, or other similar type materials.

In the illustrated embodiment, housing 200 includes a cylindrical body portion 206 that forms an annular surface with a first edge 210 and a second edge 212. Housing 200 also includes an outwardly extending flange 214 disposed proximate second edge 212 and around a perimeter of housing 200. As such, outwardly extending flange 214 forms a lip that extends from body portion 206. As will be described in more detail below, outwardly extending flange 214 is configured to secure housing 200 within fitting 204.

Housing 200 functions to house transparent protective lens 201. In particular embodiments, lens 201 may be made of glass, acrylic, Polystyrene, Polycarbonate, Polymethylmethacrylate (PMMA), and Co-Polyester. These materials are provided as examples only, however. It is recognized that lens 201 may be formed from any suitable transparent material. It is further recognized that these materials and other suitable materials may be uncoated. Alternatively, the materials may be coated with an-antireflective coating to reduce glare.

Lens 201 may be coupled within housing 200 in any suitable manner. In one embodiment, a plurality of inwardly protruding tabs 216 are utilized to secure lens 201 therein. Inwardly protruding tabs 216 are integrally formed on the interior surface of body portion 206 and are described in more detail with respect to FIG. 3. In the illustration of FIG. 2, however, it can be seen that inwardly protruding tabs 216 are disposed at an angle. As a result, when transparent protective lens 201 is disposed on or between inwardly protruding tabs 216, transparent protective lens 201 is oriented at an angle. Specifically, transparent protective lens 201 is oriented such that a geometric normal to the lens 201 forms an angle with respect to an optical axis 109 of an objective lens housed within the objective lens barrel 106 when the objective lens barrel coupling member 202 is coupled to the objective lens barrel 106. Although any suitable angle may be utilized for lens 201, the angling of lens 201 facilitates the substantial reduction or elimination of any glare encountered during a surgical procedure. Depending on the lighting within an operating room, a glare may occur to a user of microscope 102. In order to reduce or eliminate that glare, the user merely rotates housing 200 to change the angle of refraction of the light so that it does not shine into the objective lens of microscope 102 in an undesirable manner.

In one embodiment, lens 201 includes a tab 218 for facilitating removal of transparent protective lens 201 from housing 200 in the event that lens 201 becomes damaged or unusable during a surgical procedure. Tab 218 is formed integral to and from the same material as lens 201 and enables a user of microscope 102 to handle lens 201 without dirtying or smudging the portions of lens 201 that are viewed through microscope 102. To insert lens 201 within housing 200, body portion 206 includes a slot 219. Lens 201 may be inserted into slot 214 such that lens 201 is positioned between inwardly protruding tabs 216. When lens 201 is disposed within housing 200, tab 218 may extend past the outside perimeter of housing 200 such that lens 210 may be easily removed and replaced without requiring that drape lens cover system 104 or housing 200 be removed from microscope 102. Tab 218 may also help facilitate the rotating of housing 200 with respect to objective lens barrel 106.

Objective lens barrel coupling member 202 functions to couple housing 200 to objective lens barrel 106 of microscope 102. Coupling member 202 is a cylindrical ring formed out of any appropriate material and is generally of a diameter that is slightly larger than the diameter of objective lens barrel 106. In a particular embodiment, coupling member 202 may be formed out of elastomer or another flexible material. For frictionally retaining flexible coupling member 202 around objective lens barrel 106, flexible coupling member 202 includes a first retainer flange 220 formed around the perimeter of the interior side of flexible coupling member 202. First retainer flange 220 is formed proximate a first edge 222 which is disposed proximate objective lens barrel 106 when drape lens cover system 104 is coupled to microscope 102. First retainer flange 220 defines objective lens barrel aperture 203, which has a diameter slightly smaller than the diameter of objective lens barrel 106. However, when flexible coupling member 202 is made of a flexible material such as elastomer, flexible coupling member 202 may be stretched over the end of objective lens barrel 106 and first retainer flange 220 may elastically constrict about objective lens barrel 106. Accordingly, first retainer flange 220 holds objective lens barrel coupling member 202 in place around objective lens barrel 106. Although objective lens barrel aperture 203 is illustrated as being circular, it is recognized that objective lens barrel aperture 203 may have any suitable size and shape. Other suitable shapes are contemplated by the present invention.

In the illustrated embodiment, flexible coupling member 202 includes a second retainer flange 224 for retaining outwardly extending flange 214 of housing 200. Second retainer flange 224 is formed around the perimeter of the interior side of flexible coupling member 202 proximate a second edge 226, which is disposed proximate housing 200 when housing 200 is coupled to flexible coupling member 202. Second retainer flange 224 defines a housing aperture 228, which has a diameter substantially the same as the diameter of housing 200. Second retainer flange 224 forms a ledge upon which outwardly extending flange 214 of housing 200 rests when housing 200 is coupled to flexible coupling member 202. Although housing aperture 228 is illustrated as being circular, it is recognized that housing aperture 228 may have any suitable size and shape corresponding with the size and shape of housing 200.

As illustrated, coupling member 202 includes a third retainer flange 230. Third retainer flange 230 is formed around the perimeter of the interior side of flexible coupling member 202. Third retainer flange 230 may be formed at any location within the interior surface of flexible coupling member 202. In a particular embodiment, third retainer flange 230 may be formed at a midpoint location between first edge 222 and second edge 226. Third retainer flange 230 functions to separate objective lens barrel 106 from housing 200 within flexible coupling member 202. Thus, where flexible coupling member 202 includes third retainer flange 230, outwardly extending flange 214 of housing 200 may be nested between second retainer flange 224 and third retainer flange 230. By contrast, the edge of objective lens barrel 106 may next between first retainer flange 220 and second retainer flange 224.

Objective lens barrel coupling member 202 also functions to couple drape lens cover system 104 to drape 100 via an annular surface 231 disposed around a perimeter of objective lens barrel coupling member 202 proximate second edge 226. Drape 100 may be coupled to annular surface 231 in any suitable manner, such as adhesive coupling.

As described above, drape lens cover system 104 includes a fitting 204 disposed between housing 200 and flexible coupling member 202 for facilitating the rotation of housing 200 (and, thus, lens 201 within housing) with respect to objective lens barrel 106. Fitting 204 is a substantially L-shaped member having two legs. Fitting 204 may be slid down the annular surface 206 of housing 200 from first edge 210 to second edge 212. Example materials that may be used to form fitting 204 include polyethylene, acrylonitrile butadiene styrene (ABS), nylon, or other similar type materials. First and second legs 232 and 234 of fitting 204 are disposed between outwardly extending flange 214 of housing 200 and second retainer flange 224 of flexible coupling member 202. Friction prevents fitting 204 from rotating with respect to flexible coupling member 202. However, fitting 204 reduces friction between housing 200 and flexible coupling member 202 and enables housing 200 to be easily rotated within flexible coupling member 202. Thus, in operation, flexible coupling member 202 is frictionally retained about objective lens barrel 106 and is not rotatable with respect to objective lens barrel 106. Similarly, fitting 204 is frictionally retained within second retainer flange 224 of flexible coupling member 202 and may not be rotated with respect to flexible coupling member 202 and/or objective lens barrel 106. However, housing 200 is rotatable with respect to objective lens barrel 106, fitting 204 and flexible coupling member 202.

In the illustrated embodiment, fitting 204 includes a plurality of tabs 236 extending from first leg 232. Tabs 236 function to secure fitting 204 on housing 200. Tabs 236 extend over the upper edge of outwardly extending flange 214 and prevent fitting 204 from sliding down the cylindrical body portion 206 of housing 200. Additionally, tabs 236 further reduce friction that may prevent or hinder the rotation of housing 200 with respect to flexible coupling member 202. Stated differently, tabs 236 may reduce the effort required to rotate housing 200 within flexible coupling member 202. Although any suitable number of tabs may be utilized, it is generally recognized that tabs 236 are optional and may be omitted. Alternatively, tabs 236 may be replaced with a continuous annular element having any suitable dimensions.

FIG. 3 is a partial cross-sectional view showing the arrangement of the components of drape lens cover system 104 when assembled to objective lens barrel 106. Specifically, FIG. 3 illustrates the assembly of housing 200, objective lens barrel coupling member 202, fitting 204, and transparent protective lens 201 when assembled together and upon the objective lens barrel 106.

As can be seen in the illustrated embodiment, fitting 204 lies adjacent outwardly extending flange 214 such that fitting 204 is secured between second retainer flange 224 and outwardly extending flange 214. Fitting 204 is secured in an inner depression formed in objective lens barrel coupling member 202 between second retainer flange 224 and third retainer flange 230. Friction between flexible coupling member 202 (second retainer flange 224, specifically) and fitting 204 holds fitting 204 securely in place and prevents rotation of fitting 204. By contrast, fitting 204 functions as a track in which outwardly extending flange 214 may easily rotate. Thus, housing 200 is rotatably secured adjacent first leg 232 and second leg 234 of fitting 204. Locking tab 236 prevents outwardly extending flange 214 from slipping outside of the track created by first leg 232 and second leg 234.

The materials selected for the various components illustrated in FIG. 3 may provide for the functionality described above. For example, in a particular embodiment, lens housing 200 and fitting 204 may be formed of a relatively rigid material, such as plastic. Specific materials that may be used include polyethylene, acrylonitrile butadiene styrene (ABS), nylon, or other similar type materials. Lens housing 200 and fitting 204 are made of relatively rigid material in order to provide a relatively rigid structure for transparent protective lens 201 and to allow the outwardly extending flange 214 to slide freely relative to fitting 204. By contrast, objective lens barrel coupling member 202 may be formed of a elastomer or another flexible material such that inwardly protruding first retainer flange 220 of objective lens barrel coupling member 202 is configured to provide a resilient friction fit to the objective lens barrel 106. Specifically, first retainer flange 220 and/or the objective lens barrel coupling member 202 may be deformed slightly when placed over objective lens barrel with a resilient friction fit. A flexible material such as elastomer also allows for the creation of friction between the flexible coupling member 202 and fitting 204 to prevent fitting 204 from rotating within flexible coupling member 202.

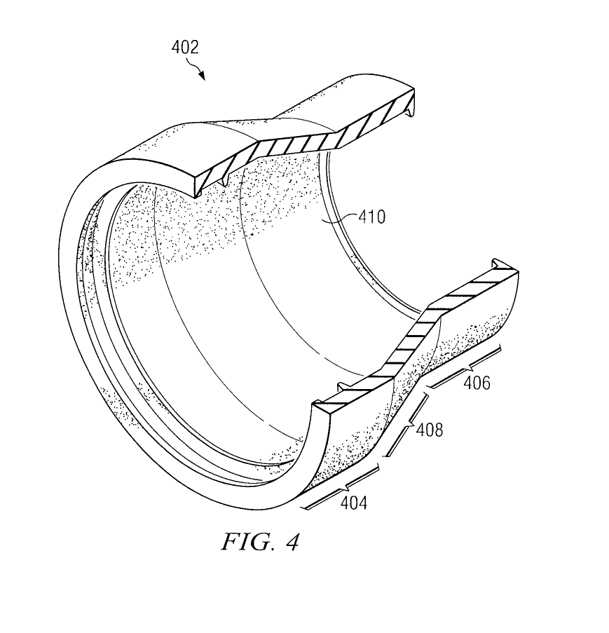

FIG. 4 is a cross-sectional view illustrating a coupling member 402 in accordance in accordance with an alternative embodiment. Although coupling member 402 is similar to coupling member 202 of FIG. 3, objective lens barrel coupling member 402 is configured to couple to an objective lens barrel that has a diameter smaller than that which is coupled to coupling member 202.

In the illustrated embodiment, coupling member 402 includes a first portion 404, a second portion 406, and a third portion 408. Each of the first, second and third portions include generally cylindrical annular surfaces. However, first portion 404 has an exterior diameter that is larger than the exterior diameter of second portion 406. In particular embodiments, the exterior diameter of first portion 404 is sufficient to define a housing aperture of an appropriate diameter for coupling to housing 200 of FIG. 1. Second portion 406 defines an interior objective lens barrel aperture 410 that is approximately the same size and shape of an objective lens barrel 106. Although a generally circular shape of objective lens barrel aperture 410 is illustrated in FIG. 4, other suitable shapes are contemplated by the present invention. Objective lens barrel aperture 410 has a diameter slightly larger than the diameter of an objective lens barrel to which it attaches (not illustrated). However, because the diameter of second portion 406 is smaller, the diameter of objective lens barrel aperture 410 may also be smaller. Accordingly, in particular embodiments, a smaller objective lens barrel may be accommodated by objective lens barrel aperture 410.

In the illustrated embodiment, coupling member 402 also includes a third portion 408. Third portion 408 is a transitioning portion that couples first portion 404 to second portion 406. The exterior diameter of third portion 408 proximate first portion 404 is the same as the exterior diameter of first portion 404. Similarly, the exterior diameter of third portion 408 proximate second portion 406 is the same as the exterior diameter of second portion 406. Thus, the outside annular surface defined by third portion 408 slopes from a larger diameter proximate first portion 404 to a smaller diameter proximate second portion 406. Although a third portion 408 is illustrated, it is generally recognized that third portion 408 may be omitted. In such an embodiment, the diameter change between first portion 404 and second portion 406 will be depicted as a step rather than as a slope.

Although embodiments of the invention and some of their advantages are described in detail, a person skilled in the art could make various alterations, additions, and omissions without departing from the spirit and scope of the present invention as defined by the appended claims.

What is claimed is:

1. A microscope drape system, comprising:
a microscope;
a drape positioned to provide a protective barrier between the microscope and a surgical environment;
a coupling member formed from a flexible material and configured to couple the drape to an objective lens barrel of a microscope;
a lens housing formed from a relatively rigid material and coupled to the coupling member, the lens housing comprising:
a cylindrical annular surface; and
an outwardly extending flange, that lies adjacent an inwardly extending flange of the coupling member;
a transparent protective lens disposed within the lens housing, the transparent protective lens positioned such that a geometric normal of the transparent protective lens forms an angle with respect to an optical axis of an objective lens housed within the objective lens barrel when the lens housing is coupled to the objective lens barrel; and
a fitting formed from a relatively rigid material and disposed between the outwardly extending flange of the housing and the inwardly extending flange of the coupling member;
wherein the fitting is frictionally retained adjacent the coupling member to prevent the rotation of the fitting relative to the coupling member; and
wherein the fitting enables the rotation of lens housing relative to the coupling member.

2. A microscope drape coupling system, comprising:
a coupling member configured to couple to an objective lens barrel of a microscope;
a lens housing coupled to the coupling member, the lens housing having a transparent protective lens disposed within the lens housing, the transparent protective lens positioned such that a geometric normal of the transparent protective lens forms an angle with respect to an optical axis of an objective lens housed within the objective lens barrel when the housing is coupled to the objective lens barrel; and
at least a portion of a fitting disposed between the coupling member and the lens housing, the fitting frictionally retained adjacent the coupling member to prevent the rotation of the fitting relative to the coupling member, the fitting enabling the rotation of lens housing with respect to the objective lens barrel coupling member.

3. The microscope drape coupling system of claim 2, wherein the lens housing has an outwardly extending flange that lies adjacent an inwardly extending flange of the objective lens barrel coupling member, the fitting disposed between the outwardly extending flange of the housing and the inwardly extending flange of the objective lens barrel coupling member.

4. The microscope drape coupling system of claim 3, wherein the fitting is an L-shaped member comprising a first leg and a second leg.

5. The microscope drape coupling system of claim 2, wherein the coupling member is of a flexible material.

6. The microscope drape coupling system of claim 5, wherein the flexible material is a polymer.

7. The microscope drape coupling system of claim 5, wherein the flexible material is a thermoplastic elastomer.

8. The microscope drape coupling system of claim 2, wherein the lens housing is of a relatively rigid material.

9. The microscope drape coupling system of claim 8, wherein the relatively rigid material is selected from the group consisting of polycarbonate, polyethylene, acrylonitrile butadiene (ABS), and nylon.

10. The microscope drape coupling system of claim 2, wherein the fitting is of a relatively rigid material.

11. The microscope drape coupling system of claim 10, wherein the relatively rigid material is selected from the group consisting of polycarbonate, polyethylene, acrylonitrile butadiene (ABS), and nylon.

12. The microscope drape coupling system of claim 2, wherein the transparent protective lens is of a material selected from the group consisting of glass, acrylic, Polystyrene, Polycarbonate, Polymethylmethacrylate (PMMA), and Co-Polyester.

13. The microscope drape coupling system of claim 12, wherein the transparent protective lens is coated with an anti-reflective coating.

14. The microscope drape coupling system of claim 2, wherein the transparent protective lens is removable from the housing.

15. The microscope drape coupling system of claim 2, wherein the transparent protective lens comprises a tab for facilitating the removal and replacement of transparent protective lens from the housing.

16. The microscope drape coupling system of claim 2, wherein the coupling member comprises an annular surface disposed around a perimeter of the coupling member, the annular surface configured to be coupled to a drape.

17. The microscope drape coupling system of claim 2, wherein an exterior surface of the coupling member comprises:
a first portion of a first diameter, the first portion defining a housing aperture; and
a second portion of a second diameter that is smaller than the first diameter, the second portion defining an objective lens barrel aperture; and
wherein the objective lens barrel aperture is smaller than the housing aperture.

18. A method of coupling a drape to a microscope, comprising:
providing a coupling member having an objective lens barrel aperture;
rotatably coupling a lens housing to the coupling member;
coupling a transparent protective lens to the housing, the transparent protective lens positioned such that a geometric normal of the transparent protective lens forms an angle with respect to an optical axis of an objective lens housed within the objective lens barrel when the housing is coupled to the objective lens barrel; and
positioning at least a portion of a fitting between the coupling member and the lens housing, the fitting frictionally retained adjacent the coupling member to prevent the rotation of the fitting relative to the coupling member; and
rotating a lens housing relative to the coupling member and the fitting.

19. The method of claim 18, wherein the lens housing has an outwardly extending flange that lies adjacent an inwardly extending flange of the objective lens barrel coupling member, the method further comprising:
positioning the fitting between the outwardly extending flange of the housing and the inwardly extending flange of the objective lens barrel coupling member.

20. The method of claim 18, wherein the fitting is an L-shaped member comprising a first leg and a second leg.

21. The method of claim 18, further comprising forming the coupling member from a flexible material.

22. The method of claim 21, wherein the flexible material is a polymer.

23. The microscope drape coupling system of claim 21, wherein the flexible material is a thermoplastic elastomer.

24. The method of claim 18, further comprising forming the lens housing from a relatively rigid material.

25. The method of claim 24, wherein the relatively rigid material is selected from the group consisting of polycarbonate, polyethylene, acrylonitrile butadiene (ABS), and nylon.

26. The method of claim 18, further comprising forming the fitting from a relatively rigid material.

27. The method of claim 26, wherein the relatively rigid material is selected from the group consisting of polycarbonate, polyethylene, acrylonitrile butadiene (ABS), and nylon.

28. The method of claim 18, further comprising forming the transparent protective lens from a material selected from the group consisting of glass, acrylic, Polystyrene, Polycarbonate, Polymethylmethacrylate (PMMA), and Co-Polyester.

29. The method of claim 28, wherein the transparent protective lens is coated with an anti-reflective coating.

30. The method of claim 18, wherein the transparent protective lens is removable from the housing.

31. The method of claim 18, wherein the transparent protective lens is comprises a tab for facilitating the removal and replacement of transparent protective lens from the housing.

32. The method of claim 18, wherein the coupling member comprises an annular surface disposed around a perimeter of the coupling member, the method further comprising:
coupling a drape to the annular surface.

33. The method of claim 18, wherein an exterior surface of the coupling member comprises:
a first portion of a first diameter, the first portion defining a housing aperture; and
a second portion of a second diameter that is smaller than the first diameter, the second portion defining an objective lens barrel aperture; and
wherein the objective lens barrel aperture is smaller than the housing aperture.
